(19) United States
(12) Patent Application Publication
(10) Pub. No.: US 2012/0178914 A1
(43) Pub. Date: Jul. 12, 2012

(54) POLYPEPTIDE MODIFICATION

(75) Inventors: Lee A. Henderson, Ithaca, NY (US); G. Scott Fletcher, King Ferry, NY (US)

(73) Assignee: VYBION, INC., Ithaca, NY (US)

(21) Appl. No.: 13/497,667

(22) PCT Filed: Sep. 21, 2010
(86) PCT No.: PCT/US10/49599

§ 371 Date: Mar. 22, 2012

Related U.S. Application Data
(60) Provisional application No. 61/245,777, filed on Sep. 25, 2009.

(51) Int. Cl. C07K 1/107; C07K 14/00

(52) U.S. Cl. 530/410

(57) ABSTRACT

The invention provides methods for the PEGylation of an N-terminal cysteine of a polypeptide such that the thiol group of the cysteine is unreacted in the fmal PEGylated polypeptide. In one embodiment, the invention comprises a method of PEGylating a polypeptide having an N-terminal cysteine, the method comprising: contacting the polypeptide with a polyethylene glycol (PEG) derivative having a free aldehyde group in a reaction mixture under reducing conditions such that the N-terminal cysteine in the resultant PEGylated polypeptide has a free thiol group.

### CROSS-REFERENCE TO RELATED APPLICATIONS

This application claims the benefit of co-pending U.S. Provisional Patent Application No. 61/245,777, filed 25 Sep. 2009, which is hereby incorporated herein.

### BACKGROUND OF THE INVENTION

Proteins and polypeptides have proved useful as therapeutics. They suffer, however, from a number of deficiencies, including a short circulating half-life, immunogenicity, susceptibility to proteolytic degradation, and low solubility. Among the strategies for reducing or eliminating these deficiencies is PEGylation, the covalent attachment of polyethylene glycol (PEG) to a protein or polypeptide. The size of the PEG attached to such a protein or polypeptide significantly affects the combined polypeptide's circulating half-life, with larger PEGs typically providing longer half-lives. The PEG moiety also increases water solubility and decreases immunogenicity.

While PEGylation of any type will generally reduce the deficiencies above, the process sometimes introduces its own drawbacks. For example, PEGylation of multiple sites of the same polypeptide can result in decreased potency of the polypeptide due to disturbance of the interaction(s) between the polypeptide and its biological target molecule(s). Multiple PEGylation of the same polypeptide will typically result in a heterogeneous mixture of the final product, resulting in PEGylated polypeptides having varying specific activities and/or requiring difficult, and often expensive, purification.

In response to these and other drawbacks of non-specific PEGylation, a number of site-specific PEGylation processes have been proposed. For example, International Patent Application Publication No. WO2007/139997 to Dong et al. describe the use of PEG-aldehyde and other PEG derivatives.

### SUMMARY OF THE INVENTION

The invention relates generally to PEGylation and, more particularly, to the PEGylation of an N-terminal cysteine residue of a polypeptide such that a PEG is covalently bound directly or via an alkylene bridge to the N-terminal amine and the thiol group of the cysteine is unreacted in the final PEGylated polypeptide. As used herein, a polypeptide is meant to include oligopeptides, polypeptides, proteins (including an antibody), and any polypeptide-containing molecule, such as a DNA/RNA-protein hybrid.

A first aspect of the invention provides a method of PEGylating a polypeptide having an N-terminal cysteine, the method comprising: contacting the polypeptide with a polyethylene glycol (PEG) derivative having a free aldehyde group in a reaction mixture. The thiol group may be either a free thiol or have an association through a disulfide. In some embodiments, a polypeptide having a thiol that is disulfide bonded is modified under reducing conditions so as to disrupt the disulfide bond.

A second aspect of the invention provides a method of improving at least one pharmaceutical or pharmacological characteristic of a polypeptide, the method comprising: reacting a polyethylene glycol (PEG) aldehyde having at least one free aldehyde group to a free α-amino group cysteine residue of the polypeptide to form an intermediate product of formula I

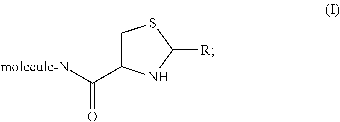

and

reducing the intermediate product with a reducing agent to yield a product of formula II

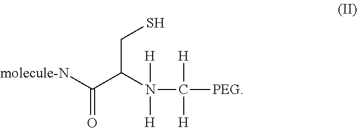

In illustrative embodiments, at least one pharmaceutical or pharmacological characteristic of the product of formula II is improved with respect to the original polypeptide.

A third aspect of the invention provides polypeptides PEGylated according to methods of the invention.

These aspects of the inventions are fully described hereinbelow. In addition, the invention comprises other aspects that are not specifically described or illustrated below but that are otherwise apparent to persons of skill in the art.

### DETAILED DESCRIPTION

The present invention includes methods for the PEGylation of an N-terminal cysteine as well as polypeptides prepared by such methods.

Methods according to embodiments of the invention comprise (1) contacting a free aldehyde group of a PEG derivative with a free α-amino group of an N-terminal cysteine residue of a polypeptide to be PEGylated, such that a 1,3-thiazolidine functional group is formed between the PEG and the polypeptide and (2) reducing the 1,3-thiazolidine to form a final polypeptide with an unreacted thiol group on the N-terminal cysteine. Polypeptides amenable to such PEGylation include oligopeptides, polypeptides, proteins, antibodies, and peptide nucleic acids (i.e., protein—DNA/RNA hybrids).

Any number of PEG-aldehydes may be used in practicing the invention, including monofunctional PEG derivatives having a single free aldehyde group and homo- or hetero-bifunctional PEG derivatives. Monomethoxy PEG (mPEG) butyraldehyde, for example, is a heterobifunctional PEG derivative having a free aldehyde group suitable for use in practicing the invention. Other useful PEG derivatives will be known to one skilled in the art.

The 1,3-thiazolidine intermediate may be reduced using any number of reducing agents. A preferred reducing agent is sodium cyanoborohydride. Other reducing agents, such as tris(carboxyethyl)phosphine (TCEP), may be used, provided they are capable of reducing the intermediate such that the 1,3-thiazolidine ring is opened and the thiol group reformed.

Such reduction of the intermediate may be achieved by maintaining a reducing environment by, for example, continually adding the reducing agent throughout the course of the PEGylation process. As used herein, continually shall mean either or both of continuous and pulsatile, i.e., intermittent, addition of the reducing agent.

In addition to methods for PEGylating N-terminal cysteine residues and polypeptides produced by such PEGylation, the invention further comprises methods of improving a pharmaceutical and/or pharmacological characteristic of a polypeptide by, for example, PEGylating an N-terminal cysteine residue of the polypeptide, as described herein. Pharmacological properties amenable to such improvement include, for example, resistance to enzymatic degradation, circulating half-life, and resistance to filtration, particularly renal filtration. Pharmaceutical properties amenable to such improvement include, for example, molecular weight and water solubility. One skilled in the art will recognize, of course, that such pharmacological and pharmaceutical properties are often linked, such that improvement of one will necessarily or likely result in improvement in the other.

In illustrative embodiments, reducing conditions are present immediately following the addition of the PEG derivative having an aldehyde group. In illustrative embodiments, reducing conditions are created promptly following mixing the PEG derivative with the polypeptide and while the conditions may vary during the reaction time, reducing agent is added continuously or intermittently during the reaction time such that the reducing conditions are maintained during most of the reaction time, e. g., 60%, 70%, 80% 90% or greater than 90% of the time. In typical embodiments of the invention, pH is maintained at about 6.8, e.g., pH 6.3-7.3. So, for example, buffer can be introduced in pulsatile manner, e.g., each time the pH reaches a threshold level, e.g., 7.3, so as to maintain pH within an optimum range. The buffer added in this way can also comprise the reducing agent.

Typically, the reaction is allowed to go to completion, which means that at least about 50%, 60% 70% or 80% of the polypeptide has been derivatized in accordance with this invention. An illustrative reaction scheme is shown below, wherein a monofunctional PEG-aldehyde is contacted with a polypeptide having an N-terminal cysteine residue and the 1,3-thiazolidine intermediate is reduced using sodium cyanoborohydride. It should be understood that the reaction scheme below is merely illustrative of one explanation of the chemical reaction. Applicants are not bound to a particular theory regarding the reaction, in whole or in part.

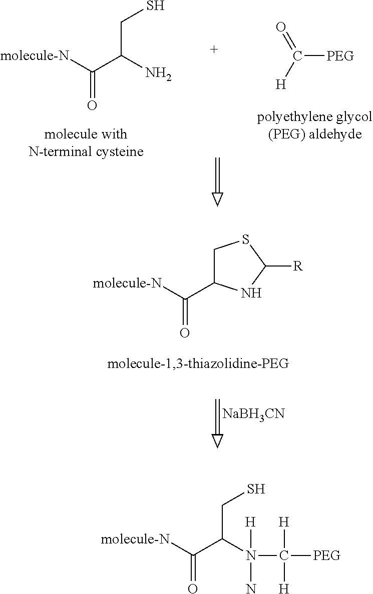

The reaction scheme above is merely illustrative. N-terminal cysteines may be PEGylated using other or additional PEG derivatives and/or reducing agents. For example, the example below exemplifies the PEGylation of a protein having an N-terminal cysteine using monomethoxy PEG butyraldehyde and sodium cyanoborohydride.

In an illustrative reaction, the polypeptide to be derivatized and the PEG aldehyde are mixed in approximately a 1:1 (PEG aldehyde: polypeptide) molar ratio at ambient temperature, approximately pH 6.8. An excess of reducing agent, e.g., a 10× molar excess, is added at the time the reaction mixture is formed and then approximately every four hours thereafter until the reaction is complete. From 5-20% additional PEG aldehyde is added, e.g., once per day, followed by 50-200 mgs of sodium cyanoborohydride, for each gram of PEG aldehyde, three or more times daily until the reaction is complete. In the final reaction mixture, the molar ratio of PEG derivative to polypeptide based on amounts added to the reaction mixture is about 2:1 to about 5:1. Progress of the reaction is measured, at least, once or twice per day, e.g., by size exclusion chromatography-high performance liquid chromatography (SEC-HPLC). A typical reaction time is about 7-14 days. The reaction is complete when at least about 70% of the polypeptide has been derivatized. The PEGylated polypeptide is then isolated such as by diafiltration and Q Sepharose chromatography (e.g., pH 10.0 to 6.8 gradient, diluted 5:1 v/v in 20 mM ethanolamine) to separate PEGylated from unPEGylated polypeptides.

The pegylation process can take up to 2 weeks and the purification (e.g., concentration and diafiltration) can take 2 weeks as well. Starting PEGylation with purified polypeptide in a phosphate buffer at neutral pH instead in lyophilized form can increase yields and lower processing costs.

### Example 1

Neoferon (Pepgen Corporation), a modified interferon-alpha-2b in development for use as an anti-viral and anti-cancer agent, was hydrated by adding 80 mL of PEGylation buffer (7 mM sodium phosphate monobasic, 18 mM sodium phosphate dibasic, pH 6.8±0.5 at 23° C.±4° C.) to 500 mg of lyophilized Neoferon, and vortexing. The hydrated Neoferon was then dialyzed to remove sucrose using a 1 kDa dialysis bag and flushing with PEGylation buffer. The resulting Neoferon (488 mg) solution was then diluted to 2.0 mg/mL with PEGylation buffer.

To the diluted Neoferon solution was added 2.0 g of mPEG2-BUTYRALD-40K [(methyl ether polyethylene glycol (20 KD))₂-CH₂CH₂CH₂CHO]. (mPEG is also referred to as methoxypoly(ethylene glycol).) The mPEG2-BUTYRALD-40K was dissolved completely and 100 mg of sodium cyanoborohydride added to the solution. Daily for 14 days, 100 mg of additional mPEG2-BUTYRALD-40 was added followed by an additional 100 mg additions of sodium cyanoborohydride three times daily for 14 days in 4 hour intervals (8 am, noon and 4 pm). Periodic aliquots were taken for SEC-HPLC analysis (7 mM monobasic sodium phosphate and 18 mM dibasic sodium phosphate pH 6.8 at 23° C.) to determine the % completion of the pegylation reaction. Then, the PEGylated polypeptide was harvested at 68% conversion.

Q Sepharose chromatography is used to separate the PEGlyated neoferon from the unpegylated neoferon and unreacted PEG derivative. The reaction mixture was diluted 1:5 in 20 mM ethanolamine pH 10.5 and the pH is adjusted to pH 10.5 with either HCl or NaOH if necessary. The diluted reaction mixture is loaded onto a Q-Sepharose column and the column is washed with 20 mM ethanolamine pH 10.5. The column is then eluted with a Phosphate buffer at pH 6.8. Unexpectedly, PEGylated neoferon does not bind to the column, but the remaining unreacted PEG derivative and unreacted neoferon did bind to the column under the conditions used.

The PEGylated Neoferon was then filtered using a 30 kDa Amicon filter and diafiltrated (137 mM sodium chloride, 2 mM acetate, 0.5% TWEEN 80, pH 6.0) to yield 184 mg.

The method above yielded about 37% of the original reconstituted Neoferon as PEGylated polypeptide.

### Example 2

3 ml of hydrated lyophilized Neoferon were prepared at 5.0 mg/ml and 1K-dialyzed into PBS pH 6.8 to remove the sucrose. After dialysis, the protein concentration was determined to be 3.3 mg/ml by Coomassie+assay. A small scale optimization experiment followed using the dialyzed Neoferon:

2:1 PEG at 1.0 mg/ml Neoferon

2:1 PEG at 2.0 mg/ml Neoferon

3:1 PEG at 1.0 mg/ml Neoferon.

NaCNBh was added at 10× molar excess.

After 1 hour, the samples were analyzed by SEC-HPLC. A very small amount of PEGylation was occurring. A decision was made to spike the samples with 2.0 mg of dry NaCNBh. The samples were mixed slowly overnight at room temperature.

Analysis by SEC HPLC then showed 30% PEGylation for both of the 2:1 PEG samples and 10% for the 3:1 PEG samples.

All 3 samples were 30K filtered and diafiltrated to remove the excess un-peglyated Neoferon.

### Example 3

75 mg of lyophilized Neoferon were resuspended in PBS pH 6.8 and 1 kDa dialyzed to remove the sucrose. 63.3 mg was recovered after dialysis and it was diluted to 2.0 mg/ml.

253.04 mg of MPEG2 BUTYRALD-40K along with 3.98 mgs of NaCNBh (10× molar excess) was added to the Neoferon. The solution was slowly mixed at room temp. At 1.7 hours the solution was analyzed by SEC-HPLC. The results showed less than 5% conversion. A decision was made to add 10 mg of NaCnBh to the solution at intervals to drive the reaction to completion. NaCNBh was added at 2:00 pm, 3:00 pm, 4:00 pm, 5:00 pm, 8:00pm, and 8:00 am the following morning. The solution was then analyzed for conversion at 12:00 pm and it showed 45% conversion. The unpegylated Neoferon was removed by Amicon 30kDa filtration and then diafiltered with PBS pH 6.8 10 times to remove the excess PEG and NaCNBh. Recovery was 26.6% of the total input Neoferon.

| | Post | | Post | | Post | | Total % |
|---|---|---|---|---|---|---|---|
| Starting | dialysis | % loss | pegylation | % loss | purification | % loss | recovery of |
| material | Neoferon | post | Neoferon | post | neoferon | post | pegylated |
| neoferon | recovered | dialysis | recovered | pegylation | recovered | purification | neoferon |
| 75 mg | 63.3 mg | 15.6% | 30 mg | 53% | 20 mg | 33% | 26.6% |

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

1. A method of PEGylating a polypeptide having an N-terminal cysteine, the method comprising:
contacting the polypeptide with a polyethylene glycol (PEG) derivative having a free aldehyde group in a reaction mixture under reducing conditions such that the N-terminal cysteine in the resultant PEGylated polypeptide has a free thiol group.

2. The method of claim 1, wherein reducing conditions are maintained substantially until the reaction is complete.

3. The method of claim 1, wherein an intermediate product of formula I is formed 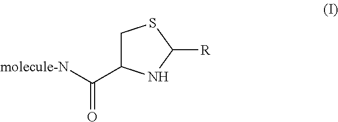
and a reduced product of formula II is formed

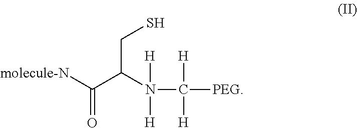

4. The method of claim 1, wherein the PEG derivative is a monofunctional PEG derivative having a single free aldehyde group.

5. The method of claim 1, wherein the PEG derivative is a bifunctional PEG derivative.

6. (canceled)

7. (canceled)

8. The method of claim 1, wherein a reducing agent is added to the reaction mixture.

9-11. (canceled)

12. The method of claim 8, wherein the reducing agent is sodium cyanoborohydride.

13. The method of claim 12, wherein the sodium cyanoborohydride is initially added at a 10:1 molar ratio, relative to the PEG derivative.

14. The method of claim 1, wherein the pH of the reaction mixture is adjusted to and maintained at about pH 6.3 to about pH 7.3 during the contacting step.

15. The method of claim 1, wherein the polypeptide is selected from the group consisting of: an oligopeptide, a polypeptide, a protein, an antibody, and a polypeptide-containing molecule.

16. The method of claim 14, wherein the polypeptide is a lyophilized protein.

17. The method of claim 1, further comprising:
removing sucrose from the lyophilized protein prior to contacting it with the PEG derivative.

18. A method of improving at least one pharmaceutical or pharmacological characteristic of a polypeptide, the method comprising:
reacting a polyethylene glycol (PEG) derivative having at least one free aldehyde group to a free a-amino group cysteine residue of the polypeptide to form an intermediate product of formula I 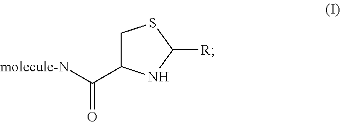
and
reducing the intermediate product with a reducing agent to yield a product of formula II 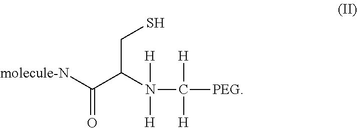
wherein at least one pharmaceutical or pharmacological characteristic of the product of formula II is improved with respect to the polypeptide.

19. The method of claim 18, wherein the pharmacological property is selected from a group consisting of: resistance to enzymatic degradation, circulating half-life, and resistance to renal filtration.

20. The method of claim 18, wherein the pharmaceutical property is selected from a group consisting of:
molecular weight and water solubility.

21. The method of claim 18, wherein the PEG derivative is a monofunctional PEG derivative having a single free aldehyde group.

22. The method of claim 18, wherein the PEG derivative is a bifunctional PEG derivative.

23. (canceled)

24. (canceled)

25. The method of claim 18, wherein reducing includes adding the reducing agent periodically to achieve a pulsed reduction.

26. The method of claim 18, wherein the reducing agent is sodium cyanoborohydride.

27. (canceled)

28. A PEGylated polypeptide with an N-terminal cysteine comprising a PEG derivative covalently bound to the amino group of the N-terminal cysteine as depicted in Formula II

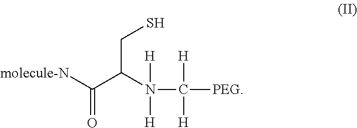

29. (canceled)

30. (canceled)
